(19) United States
(12) Patent Application Publication
(10) Pub. No.: US 2023/0055962 A1
(43) Pub. Date: Feb. 23, 2023

(54) METHODS AND SYSTEMS FOR PROVIDING RHYTHMIC SENSORY STIMULUS FOR ADMINISTRATION WITH EXTERNAL NEUROMODULATION

(71) Applicant: Wave Neuroscience, Inc., Newport Beach, CA (US)

(72) Inventors: James William Phillips, Fountain Valley, CA (US); Alexander Ring, Newport Beach, CA (US)

(21) Appl. No.: 17/821,141

(22) Filed: Aug. 19, 2022

Related U.S. Application Data
(60) Provisional application No. 63/235,013, filed on Aug. 19, 2021.

(51) Int. Cl. A61N 2/12; A61N 2/00; A61M 21/00

(52) U.S. Cl. CPC A61N 2/12; A61N 2/002; A61N 2/006; A61M 21/00; A61M 2021/0027; A61M 2021/0044; A61M 2021/0022; A61M 2209/088; A61M 2230/10

(57) ABSTRACT

Exemplary embodiments described herein provide systems and methods for administer multiple stimulus to a patient in order to improve the efficacy of the administered stimulus on the brain activity of the patient. Exemplary embodiments described herein may combined magnetic stimulation along with sensory stimulation to influence the intrinsic frequency of an EEG band of a person.

### RELATED APPLICATIONS

This application claims the benefit of U.S. Provisional Patent Application No. 63/235,013, filed Aug. 19, 2021, the contents of which are herein incorporated by reference in their entirety.

### BACKGROUND

It has been shown that imparting electrical energy noninvasively to the brain can have a significant effect on mental state, including improving performance (memory, concentration, etc.) and the alleviation of symptoms of mental disorders (major depression, PTSD, anxiety, etc.). Much speculation has been made as to possible mechanism of action, but due to the complexity of mental disorders and the inability to sense in vivo neuronal firing to any great extent, no consensus has been reached yet.

Electrical energy can be imparted directly using electrodes on the scalp (Cranial Electrical Stim, or CES), or through induction via variable magnetic fields (Transcranial Magnetic Stimulation, or TMS, or by moving permanent magnets). Other means of brain stimulation are available as well, such as focused ultrasound or pulsed light. Due to the skull's naturally high impedance to electric current flow, CES tends to provide electrical stimulation that is lower energy and more distributed than TMS, which allows TMS to target specific cortical structures (such as the dorsolateral prefrontal cortex, or DLPFC) with higher stimulation intensities, to which CES cannot easily compare.

Transcranial Direct Current Stimulation (tDCS) is one of the simplest forms of brain stimulation, in which a small (1-2 mA) constant DC electric current is imparted between two electrodes on the scalp for a period of time (usually 20-30 minutes). The electrode location is generally chosen near the pre-frontal cortex, with electrodes on either side of the forehead or near the temples, in order to impart as much current as possible through the brain, while avoiding the hair and other areas of the scalp that can increase electrode impedance or cause discomfort.

There is no clear agreed upon mechanism of action for tDCS. It is believed that it depolarizes or hyperpolarizes neurons slightly, which may encourage or discourage firing, depending on whether polarization is anodic (+) or cathodic (−).

A significant amount of research has been performed on tDCS, due mainly to its low cost and ease of use. It has shown to have a modest effect on symptoms of several psychiatric disorders, including fibromyalgia, depression, anxiety, PTSD, and schizophrenia. Data is mixed, however, with many reports showing little to no effect on cognition or memory.

Transcranial Alternating Current Stimulation (tACS) (also called Cranial electrotherapy stimulation) is similar to tDCS in that electric current is imparted to the scalp. However, tACS uses a variable current, and therefore allows for much more configurability for the user. tACS usually creates the waveform by imparting short pulses at varying amplitudes over time.

tACS has shown some benefit for treatment of depression and insomnia, and has been suggested for headaches, fibromyalgia, smoking cessation, and opiate withdrawal. However, like tDCS the results are modest and evidence is mixed. In January 2020, tACS was classified by FDA as a Class II medical device to treat insomnia and anxiety. tACS is classified by FDA as Class III for depression.

Electroconvulsive Therapy (ECT) is an extreme form of tACS, in which the amplitude of stimulation is increased to a level that causes the person to experience severe convulsions or seizures, similar to what is seen in epilepsy. ECT brings about many neuro-physiological and neuro-chemical changes in the brain. Changes occur to the expression of genes, functional connectivity, permeability of blood-brain-barrier, and alteration in the person's immune system. All these changes work together to alter the fundamental communication in the brain. The convulsions increase brain metabolism to a very high level, resulting in oxygen deprivation, which can result in memory loss and other adverse effects. ECT is approved by FDA for depression, and more than 80,000 treatments are administered in the US each year. ECT has a terrible stigma, related to the way it has been portrayed in TV and movies. However, modern ECT is quite effective, and adverse effects are relatively minor. ECT can be administered as a sinusoidal, bi-phasic, or monophasic pulsed stimulation, as shown below.

Repetitive Transcranial Magnetic Stimulation (rTMS) consists of a train of electromagnetic pulses generated by a coil held above the scalp over a target brain region. The pulses from the coil induce electric current pulses in the brain. rTMS has been cleared by FDA for treatment of major depression and Obsessive-Compulsive Disorder (OCD).

Standard rTMS provides pulses at 10 Hz, with amplitudes of about 1.5 Tesla. The pulse itself has a sinusoidal shape with a period of about 300 μsec. The mechanism of action for rTMS is poorly understood. It is thought that rhythmic pulses can alter synaptic plasticity, excite and inhibit cortical function, and provide network entrainment effects. Generally, stimulation is applied over the dorsolateral pre-frontal cortex, allowing for changes in the region generally associated with mood.

Over the years, magnetic field treatment has grown in popularity as a method of treating physical and mental disorders. As popularity for this type of treatment grows, it has shown that applying alternating magnetic fields at specific frequencies upon a user produced therapeutic and advantageous effects. For example, the follow patents and patent publications describe systems and methods for generating and/or applying a magnetic field to a patient: U.S. Pat. Nos. 9,015,057; 8,480,554; 8,926,490; 8,585,568; 9,446,259; 10,065,048; 8,456,408; 9,713,729; 8,870,737; 8,475,354; 8,888,672; 8,888,673; 8,961,386; 9,308,387; 9,272,159; 9,649,502; 9,962,555; 10,350,427; 9,308,385; 10,029,111; 10,342,986; 10,398,906; 10,420,953; 10,420,482; US 2017/0281035; US 2017/0296837; US 2018/0104504; US 2016/0045756; and US 2018/0229049, each of which is incorporated by reference in their entirety herein.

For brain stimulation, the main parameters that can be controlled are amplitude, frequency, waveform type, and location. Location is generally specified using a functional scan of the brain to determine a region of functional deficit or interference. These scans may be generated using an electroencephalogram (EEG) or functional Magnetic Resonance Imaging (fMRI) (other methods, such as magnetoencephalography (MEG), single photon emission computed tomography (SPECT), function near-infrared (fNIR) stimulation and positron emission tomography (PET) are less common).

Amplitude of stimulation is often based on safety, patient comfort, and limitations of the technology. The general rule is that the lowest amplitude possible is used that will still achieve a desired outcome.

Frequency of stimulation refers generally to alternating or pulsatile energy imparted to the brain at a specific pulse frequency. For example, CES may impart a series of very short electric pulses to the scalp, and TMS may generate a train of magnetic pulses that induce brief electric current in the brain, where the pulse frequency is set to a specific value.

Stimulation waveform type is sometimes chosen to control the energy content of an electric pulse. For example, a mono-phasic pulse resembling a square wave may have very different energy content than a bi-phasic sinusoidal pulse.

Rhythmic photic stimulation has been shown to have an effect on brain activity, implemented by flickering or flashing lights within specific frequency ranges. Specifically, flickering photic stimulation at a person's intrinsic alpha frequency (IAF) has been shown to alleviate pain or stress and may also improve behavioral performance (Kim, 2016) (Nomura, 2006) (Williams, 2006). Flickering lights tend to trigger EEG activity at frequencies at or near the photic stimulation frequency, resulting in resonant peaks in the EEG band at the stimulation frequency (Fedotchev, 1990). In addition, peaks in the EEG spectra are sometimes found at slightly higher frequencies and sometimes at subharmonic frequencies, but these are generally far lower than the peak of the dominant EEG waveform in the band (Sato, 1961). The brain's reaction as seen in the EEG to photic stimulation in the alpha range will often generate an increased rhythmic response within the alpha EEG range (Scheuler, 1983). Rhythmic stimulation at alpha frequencies has also been known to produce antidepressant-like effects (Kim, 2016)
Kim, S., Kim, S., Khalid, A., Jeong, Y, Jeong, B., Lee, S.-T., Jung, K.-H., Chu, K., Lee, S. K., & Jeon, D. (2016). Rhythmical photic stimulation at alpha frequencies produces antidepressant-like effects in a mouse model of depression. PLoS ONE, 11(1), Article e0145374.
Sato, K., Sonoda, T., Nishikawa, T., & Mimura, K. (1961) Some observations on EEG response to photic flicker stimulation. Acta Med. Nagasaki, 5, 188-196
Nomura T, Higuchi K, Yu H, Sasaki S, Kimura S, Itoh H, et al. (2006) Slow-wave photic stimulation relieves patient discomfort during esophagogastroduodenoscopy. J Gastroenterol Hepatol 21: 54-58. pmid:16706812,
Williams J, Ramaswamy D, Oulhaj A (2006) 10 Hz flicker improves recognition memory in older people. BMC Neurosci 7: 21. pmid:16515710.
Fedotchev A I, Bondar A T, Konovalov V F. Stability of resonance EEG reactions to flickering light in humans. Int J Psychophysiol. 1990 September; 9(2):189-93. doi: 10.1016/0167-8760(90)90073-m. PMID: 2228753.
Scheuler W. Zur klinischen Bedeutung der gesteigerten Photostimulationsreaktion im alpha-Frequenzbereich [Clinical significance of increased reaction to photostimulation in the alpha frequency range]. EEG EMG Z Elektroenzephalogr Elektromyogr Verwandte Geb. 1983 September; 14(3):143-53. German. PMID: 6414803.

### SUMMARY OF THE INVENTION

The present disclosure relates to rhythmic sensory stimulus provided during, or proximate to (either before or after) administration of external neuromodulation. Exemplary embodiments of the administration of rhythmic sensory stimulus may comprise administering a sensory stimulus at a periodic rhythm. The frequency of the rhythmic sensory stimulus may be equal to or approximate to the frequency of the magnetic field administered by the external neuromodulution. In an exemplary embodiment, the rhythmic sensory stimulus has a frequency, such as a pulse frequency, that may be approximately equal to, or is a harmonic or sub-harmonic of, an intrinsic frequency of an EEG band. For example, the intrinsic frequency could be the alpha frequency and the EEG band could be the Alpha band.

Exemplary embodiments provided herein may include different rhythmic sensory stimulation. For example, rhythmic stimulation could be visual, audio, or tactile, and may be consciously felt or subconsciously felt.

Exemplary embodiments may include rhythmic stimulation paired with additional sensory modifiers that change with type of sensory stimulation. Exemplary embodiments of the rhythmic stimulation disclosed here may be administered at a frequency or modulated such that a frequency is created and/or adjusted so that it is approximately equal to, or is approximately a harmonic or subharmonic of, a target frequency. The target frequency may be the magnetic frequency of the external neuromodulation and/or an intrinsic frequency of an EEG band of the patient.

Examples of rhythmic visual stimulation include, without limitation, a flashing light, moving video, alternately changing colors, changing light intensity, and alternately changing focus.

Examples of audio stimulation may include, without limitation, beeping sound, ticking sound, tick-tock sound, music, tones, voice, our nature sounds. Music could be modulated such that the beats per minute is adjusted so that it is approximately equal to, or is approximately a harmonic or subharmonic of, a target frequency, such as the magnetic frequency of the external neuromodulation and/or an intrinsic frequency of an EEG band of the patient.

Examples of tactile stimulation may include, without limitation, tapping on the skin, brushing the skin, blowing on the skin, temperature fluctuations, or administering small electric shocks.

Additional sensory modifiers may include, without limitation, different scents that may be paired with different modulated music, or the participant may be sat in a chair that rotates or leans at different angles during stimulation so as to cause the feeling of imbalance.

Examples of external neuromodulation include, without limitation, repetitive transcranial magnetic stimulation (rTMS), low-field magnetic stimulation (LFMS), neuro-EEG synchronization therapy (NEST) using rotating permanent magnets, transcranial AC stimulation (tACS), transcranial DC stimulation (tDCS), transcranial photobiomodulation, or focused ultrasound.

The present disclosure relates to magnetic field treatment technology. Specifically, to magnetic stimulation including the application of a magnetic field. The magnetic stimulation may be at a specific or variable frequency. The specific frequency may be, for example, a given frequency or may be approximately equal to, or is approximately a harmonic or subharmonic of, a target frequency, such as an intrinsic frequency of an EEG band of a patient.

The various embodiments of the present magnetic stimulation apparatus and method and/or rhythmic sensory stimulus apparatus and method have several features, no single one of which is solely responsible for the desirable attributes provided herein. Without limiting the scope of the present embodiments as expressed by the claims that follow, the more prominent features will be discussed briefly. After considering this discussion, and particularly after reading the section entitled “Detailed Description,” one will understand how the magnetic stimulation apparatus and method of the present embodiments can be used in various combinations to provide the advantages described herein.

In an exemplary embodiment, a magnetic stimulation apparatus and methods include providing a magnetic field to a patient in conjunction or in proximity to providing a rhythmic sensory stimulus to the patient.

Exemplary embodiments may include a method and devices where a rhythmic stimulus is provided. Rhythmic sensory stimulation may be imparted to a person in order to affect the brain. The stimulation could be of many different types, comprising visual (such as photic, video, etc.), audio, tactile (such as pressure), or any combination thereof. In addition, more than one type of stimulation may be combined together, such as different more than one visual, audio, tactile, or other stimulation. The sensory nerves have a direct connection to the brain, and the rhythmic firing of the sensory nerves may encourage the firing of neurons in the brain at the frequency of stimulation.

### BRIEF DESCRIPTION OF THE DRAWINGS

FIG. 1 shows one example system in which a visual and auditory stimulus may be administered in conjunction with a magnetic stimulus using a rotating permanent magnet system.

FIG. 2 shows one example system in which a visual stimulus may be administered in conjunction with a magnetic stimulus using a magnetic coil system.

FIG. 3 shows one example system in which a visual and tactile stimulus may be administered in conjunction with a magnetic stimulus.

FIGS. 4A-4F illustrate exemplary EEG signals, transformed EEG signals, and brain activity scans during exemplary embodiments described herein using rhythmic sensor and without rhythmic sensory stimulus.

FIGS. 5A-5F illustrate exemplary EEG signals, transformed EEG signals, and brain activity scans during exemplary embodiments described herein using rhythmic sensor and without rhythmic sensory stimulus.

FIG. 6 shows an example of the Q-factor as used in this invention.

FIG. 7 shows an example system of the present invention in which a person wears an EEG cap, which is electrically connected with a processing unit.

FIG. 8 shows an example system of the present invention in which a person wears a headband that comprises two or more EEG electrodes.

FIG. 9 shows an example system of the present invention in which a person wears an EEG headband, which is connected electrically to a processing unit, which comprises an EEG amplifier, a processor, and memory in order to cycle through RSFs in or around the desired EEG band, and to find the RSF which optimizes a function of the EEG recorded while the stimuli is being delivered, and it uses this optimized RSF to estimate the intrinsic frequency of the EEG band.

### DETAILED DESCRIPTION OF THE EMBODIMENTS

Example devices, methods, and systems are described herein. Any example embodiment or feature described herein is not necessarily to be construed as preferred or advantageous over other embodiments or features. The example embodiments described herein are not meant to be limiting. It will be readily understood that certain aspects of the disclosed devices, systems, and methods can be arranged and combined in a wide variety of different configurations, all of which are contemplated herein. Accordingly, any feature, component, concept, or function may be duplicated, removed, combined, or otherwise used alone or in combination with any other combination of other features, components, concepts, or functions described herein or otherwise known to a person of skill in the art. For the avoidance of doubt, for example, any combination described herein may include the permanent magnet system of the neuromodulation stimulation system, and/or may include the coil configuration without departing from the scope of the invention. As another example, any combination described herein may include any combination of rhythmic sensory stimulation as would be apparent to those of skill in the art. Exemplary combinations are shown and described, but any combination of the stimulation components and/or methods may be included in any combination, including combinations of more than two rhythmic stimulation devices or methods.

The particular arrangements shown in the figures should not be viewed as limiting. It should be understood that other embodiments might include more or less of each element shown in a given figure. Further, some of the illustrated elements may be combined or omitted. Yet further, an example embodiment may include elements that are not illustrated in the figures.

Exemplary embodiments described herein may be used combined magnetic stimulation with sensory stimulation to influence the intrinsic frequency of an EEG band of a person. Combining stimulation makes use of the resonant properties of the brain to improve brain function and to reduce the symptoms of mental disorders, to a degree above what magnetic stimulation or sensory stimulation is capable of producing on its own. By increasing the stimulation effect, it may be possible to reduce the time required for a person to undergo stimulation, making the treatment much more convenient, accessible, efficacious, and comfortable for the patient. It may also be possible to reduce the intensity of either the magnetic field or the sensory stimulation and still realize an effect, due to the additive nature of the two modalities combined, contributing to enhanced interventional safety profiles.

External neuromodulation of the brain has shown efficacy in the treatment of mental disorders. When the pulse frequency is set to influence an intrinsic frequency of an EEG band, the effect may be improved. This improvement is based upon the natural resonance of the brain. However, efficacy of neuromodulation is still limited for certain disorders. This may be due to the fact that neuromodulation generally provides stimulation to a relatively small percentage of the brain. In order to increase the effect of neuromodulation, it may be advantageous to use a complimentary rhythmic sensory stimulus, which may combine with the neuromodulation to affect a larger percentage of the brain.

Exemplary embodiments described herein include a method and device where a rhythmic sensory stimulus is provided during, or shortly before or after, administration of external neuromodulation. As used herein, shortly, or in proximity to may be within minutes of administration of one stimulus in relation to another stimulus or in a specific duration in which the effect of one stimulus may be additive to the effect of the other stimulus.

Exemplary embodiments described herein may administer the rhythmic sensory stimulus at a frequency that is equal to or approximately equal to, or is a harmonic or sub-harmonic of the pulse frequency of the external neuromodulation. When this occurs, an additive effect of the combined stimulation is achieved, allowing for significantly greater changes in the function of the brain, when compared to external neuromodulation on its own.

In an exemplary embodiment, the pulse frequency is approximately equal to, or is a harmonic or sub-harmonic of, an intrinsic frequency of an EEG band. For example, the intrinsic frequency could be the alpha frequency and the EEG band could be the Alpha band.

Exemplary embodiments described herein may include devices and methods for administering a rhythmic stimulation. Exemplary embodiments of the rhythmic stimulation may be visual, audio, tactile, or combinations thereof. Exemplary embodiments of the rhythmic stimulation may be consciously felt or subconscious. Rhythmic stimulation could be paired with additional sensory modifiers that change with type of sensory stimulation.

Examples of rhythmic visual stimulation may include, without limitation, a flashing light, moving video, alternately changing colors, changing light intensity, alternately changing focus, and combinations thereof.

Examples of audio stimulation may include, without limitation, beeping sound(s), ticking sound(s), tick-tock sound(s), music, tones, voice, nature sounds, beats, instrumental rhythm, or combinations thereof. Music could be modulated such that the beats per minute are adjusted so that it is approximately equal to, or is approximately a harmonic or subharmonic of, an intrinsic frequency of an EEG band.

Examples of tactile stimulation may include, without limitation, tapping on the skin, brushing the skin, blowing on the skin, temperature fluctuations, administering small electric shocks, or combinations thereof.

Additional sensory modifiers may include, for example, different scents that may be paired with different modulated music, or the participant may be sat in a chair that rotates or leans at different angles during stimulation so as to cause the feeling of imbalance.

Examples of external neuromodulation may include, without limitation, repetitive transcranial magnetic stimulation (rTMS), low-field magnetic stimulation (LFMS), neuro-EEG synchronization therapy (NEST) using rotating permanent magnets, transcranial AC stimulation (tACS), transcranial DC stimulation (tDCS), transcranial photobiomodulation, or focused ultrasound.

Stimulation near an intrinsic frequency of an EEG band may affect the rhythmicity of brain activity, improving network connectivity metrics, comprising coherence, and potentially shifting the intrinsic frequency higher or lower, toward a target frequency. The rhythmicity may be quantified using Q-factor, which is seen in communications and signal processing literature to estimate the relative frequency bandwidth of a signal. Low Q-factor indicates a wide bandwidth, whereas high Q-factor indicates a narrow bandwidth. A higher Q-factor is seen in the EEG of a rhythmic brain with higher coherence. Low Q-factor has been found in mental disorders such as PTSD, ADHD, autism, Parkinson's disease, drug addiction, traumatic brain injury, and fibromyalgia. By making the brain activity of people affected by disorders more rhythmic and coherent, it may be possible to reduce the symptoms of those disorders. In addition, by making the brain more rhythmic and coherent, it may be possible to improve athletic or academic performance.

Exemplary embodiments described herein may also include a device, including, any combination of and without limitation, an external neuromodulation means, and a rhythmic stimulation means using the methods and components described herein. In a preferred embodiment, the neuromodulation device may be an rTMS system using rotating magnets, and the rhythmic sensory stimulus may be a flashing light unit. In this, the flashing light unit and the rTMS may have the same controller unit, which specifies the frequency of the NEST and the flashing light. In an alternate embodiment, the flashing light unit may be positioned in close proximity to the magnetic field of the rTMS, and the magnetic field may be sensed using a magnetic sensor, such as a Hall Effect sensor, and the flashing light unit may comprise a processor, which detects the frequency or phase of the magnetic field, and adjusts the flashing light based upon the sensed parameter. Such a flashing light unit may be attached to the rTMS device, so that the flashing light may shine such that the subject can see the light, a reflection, or the aura of the light. An alternate embodiment may comprise glasses, in which an LED is attached to the glasses lens or frame, such that the light can be sensed by the person.

Exemplary embodiments of the magnetic stimulation system described herein may be conventional rTMS devices for administering a magnetic field to a patient, using coils and/or permanent magnets. Exemplary embodiments of the magnetic stimulation system described herein may include systems and devices for administering a pulse frequency magnetic field that may be individually targeted to the person's EEG and ECG. The waveform may be the same as conventional rTMS. However, by administering pulses at the brain's intrinsic frequency, at stimulation intensities that are below those normally applied in rTMS, it is thought that exemplary embodiments of rTMS described herein uses the brain's natural resonance to achieve significantly better outcomes than could normally be achieved using standard one-size-fits-all rTMS.

Exemplary embodiments of the magnetic stimulation system according to embodiments described herein may include a set of 3 rotating permanent magnets, with the frequency of rotation set to the brain's intrinsic resonance frequency. Exemplary embodiments of the waveform of the magnetic stimulation system's magnetic field may be purely sinusoidal, and stimulation is continuous. Exemplary embodiments may also or alternatively administer the magnetic stimulation for only a few seconds per minute.

Exemplary embodiments of the magnetic stimulation's system magnetic field strength may be similar to standard rTMS. However, the induced electric field may be very low, due to the lower amplitude of the sinusoidal waves compared to the very short, high voltage, rTMS pulses. Due to the resonant property of the brain at its intrinsic frequency, exemplary embodiments of the magnetic stimulation device according to embodiments described herein may still able to have a significant effect on brain activity and neuroplasticity, resulting in significant reduction in symptoms of mental disorders such as depression and PTSD.

The standard technique of finding the intrinsic alpha frequency (IAF) traditionally involves recording EEG while the subject is resting, alert with eyes closed (Valipour, 2014). The IAF may be found as the peak of the Fast Fourier Transform (FFT) between 8-13 Hz, although it may alternately be defined as the peak between 8-12 Hz. Alpha activity is often dominant in the posterior region of the brain, such that it is generally referred to as the posterior dominant rhythm. It is often highly symmetrical.

Exemplary embodiments described herein may include systems and methods for treating a subject having an intrinsic frequency in a specified EEG band. The system may be configured to and the method may include any combination of: adjusting the frequency of an alternating magnetic field based on the subject's intrinsic frequency in the specified EEG band; and applying said magnetic field close to a head of the subject; and applying said sensory stimulation near the person; and moving the intrinsic frequency toward a pre-selected intrinsic frequency within the specified EEG band using said magnetic field and sensory stimulation.

The system and method may also include adjusting a non-magnetic rhythmic sensory stimulation wherein the rhythmic frequency is equal to the frequency of the alternating magnetic field or is equal to a harmonic or subharmonic of the frequency of the alternating magnetic field. The frequency will be considered equal to another frequency if the frequency is approximately equal to another frequency such that the frequencies are within normal tolerances for the administered procedure, and/or the equipment used to select and administer a target frequency.

Exemplary embodiments described herein may include systems and methods for treating a subject having an intrinsic frequency in a specified EEG band, that may include any combination of: applying a magnetic field close to a head of the subject; and applying a sensory stimulation near the person; and moving a Q-factor of the intrinsic frequency toward a pre-selected Q-factor of the intrinsic frequency within the specified EEG band using said magnetic field and sensory stimulation.

Exemplary embodiments of the systems and methods described herein may also include any combination of: adjusting the frequency of an alternating magnetic field based on the subject's intrinsic frequency in the specified EEG band; and adjusting a non-magnetic rhythmic sensory stimulation wherein the rhythmic frequency is equal to or a harmonic or subharmonic of the frequency of the alternating magnetic field.

Exemplary embodiments described herein may include systems and methods whereby adjusting the frequency of the alternating magnetic field or of the magnetic pulse is accomplished automatically by the controller such that the frequency is equal to or a harmonic or subharmonic of the frequency of the alternating magnetic fields. Alternatively, the frequency may adjusted by input from an operator, such as by adjusting a dial, inputting a specific frequency, pressing a button to increase or decrease the frequency, or the like. Whether the adjustment is made automatically or manually, in a preferred embodiment, the output of the rhythmic frequency is controlled by the controller.

Exemplary embodiments described herein may include systems and methods of treating a subject having intrinsic frequencies among multiple sites in a brain of the subject within a specified EEG band including any combination of: providing a pre-specified coherence value; determining a coherence value of the intrinsic frequencies among multiple sites in the brain of the subject within a specified EEG band; adjusting an output of one or more magnetic fields; applying said magnetic fields close to a head of the subject; adjusting a non-magnetic rhythmic sensory stimulation wherein the rhythmic frequency is equal to or a harmonic or subharmonic of the frequency of the alternating magnetic fields; applying said sensory stimulation near the person; and moving the coherence value toward the pre-selected coherence value using said magnetic fields and sensory stimulation.

Exemplary embodiments may include systems and methods of treating a subject having an intrinsic frequency in a specified EEG band, including any combination of: adjusting the frequency of electric stimulation pulses based on the subject's intrinsic frequency in the specified EEG band; and applying said electric stimulation to a head of the subject; and adjusting a separate rhythmic sensory stimulation wherein the rhythmic frequency is equal to or a harmonic or subharmonic of the frequency of the electric stimulation; and applying said sensory stimulation near the person; and moving the intrinsic frequency toward a pre-selected intrinsic frequency within the specified EEG band using said electric and sensory stimulation.

Exemplary embodiments described herein may include systems and methods of treating a subject having an intrinsic frequency in a specified EEG band, including any combination of: adjusting the frequency of electric stimulation pulses based on the subject's intrinsic frequency in the specified EEG band; applying said electric stimulation to a head of the subject; adjusting a separate rhythmic sensory stimulation wherein the repetition frequency is equal to or a harmonic or subharmonic of the frequency of the electric stimulation; applying said sensory stimulation near the person; moving a Q-factor of the intrinsic frequency toward a pre-selected Q-factor of the intrinsic frequency within the specified EEG band using said electric and sensory stimulation.

Exemplary embodiments described herein include systems and methods of treating a subject having intrinsic frequencies among multiple sites in a brain of the subject within a specified EEG band including any combination of: providing a pre-specified coherence value; determining a coherence value of the intrinsic frequencies among multiple sites in the brain of the subject within a specified EEG band; adjusting an output of one or more electric stimulation pulse generators; applying said electric stimulation pulses close to a head of the subject; adjusting a separate rhythmic sensory stimulation wherein the rhythmic frequency is equal to or a harmonic or subharmonic of the frequency of the electric stimulation; applying said sensory stimulation near the person; and moving the coherence value toward the pre-selected coherence value using said electric and sensory stimulation.

Any of the exemplary systems and methods described herein may include the sensory stimulation comprising any combination of and/or at least one of flashing light, alternating light intensity, alternating light color, rhythmic video images, rhythmic audio sounds, music, rhythmic tapping, rhythmic electrical stimulation, rhythmic pressure pulses, and rhythmic temperature fluctuation.

Exemplary embodiments of the systems and methods described herein may include a device for generating a magnetic field using a Transcranial Magnetic Stimulation (TMS) coil.

Exemplary embodiments of the systems and methods described herein may include a device for generating a magnetic field using moving at least one permanent magnet.

Exemplary embodiments described herein include a device including any combination of: at least one permanent magnet having a rhythmic movement with a movement frequency; and at least one light having a rhythmically changing intensity with an intensity frequency; and a controller coupled to the magnet and to the light; wherein the controller may comprise a processor that controls the rhythmic movement frequency of at least one said magnet between about 0.5 Hz and 100 Hz based on an intrinsic frequency of a brain of a subject within a specified EEG band; and controls the intensity frequency of at least one light set about equal to the rhythmic movement frequency, or a harmonic or subharmonic thereof.

Exemplary embodiments described herein include a device including any combination of: at least one electric current pulse generator having a programmable pulse frequency; at least one light having a rhythmically changing intensity with an intensity frequency; and a controller coupled to the pulse generator and to the light; wherein the controller may comprise a processor that controls the pulse frequency between about 0.5 Hz and 100 Hz based on an intrinsic frequency of a brain of a subject within a specified EEG band; and controls the intensity frequency of at least one light set about equal to the pulse frequency, or a harmonic or subharmonic thereof.

As used herein, the term “intensity frequency” is used to indicate that the light may change intensity with a specified period. For example, the light used in this invention may not turn on and off or strobe, but may instead become perceptibly brighter or dimmer in accordance with the pulse frequency. Alternatively, the intensity frequency may refer to the lights ability to change color in accordance with the pulse frequency.

Exemplary devices described herein may also or alternatively include any combination of: an EEG amplifier; a operable to detect electrical brain activity; and a second electrode operable to detect a reference signal; wherein the processor may comprise logic which calculates the intrinsic frequency of the EEG band.

Exemplary embodiments of the system and method described herein may combine rhythmic sensory stimulus with external neuromodulation in order to bring about an additive effect of both modalities in order to treat the brain of a person.

Exemplary embodiments of the rhythmic sensory stimulation may include photic, audio, tactile, visual, thermal, and pressure stimuli.

Exemplary embodiments of the external neuromodulation may include repetitive transcranial magnetic stimulation (rTMS), variations of the rTMS system such as administration at an intrinsic frequency of a patient, neuro-EEG Synchronization Therapy (NEST), cranial electrical stimulation (CES), transcranial Direct Current Stimulation (tDCS), transcranial Alternating Current Stimulation (tACS), pulsed scalp photic stimulation (photobiomodulation), and focused ultrasound.

Exemplary embodiments may permit each of (or any combination of) the rhythmic sensory stimulation modalities and neuromodulation modalities to provide an additive effect on the brain when combined together, either occurring at the same time, or serially, or sequentially.

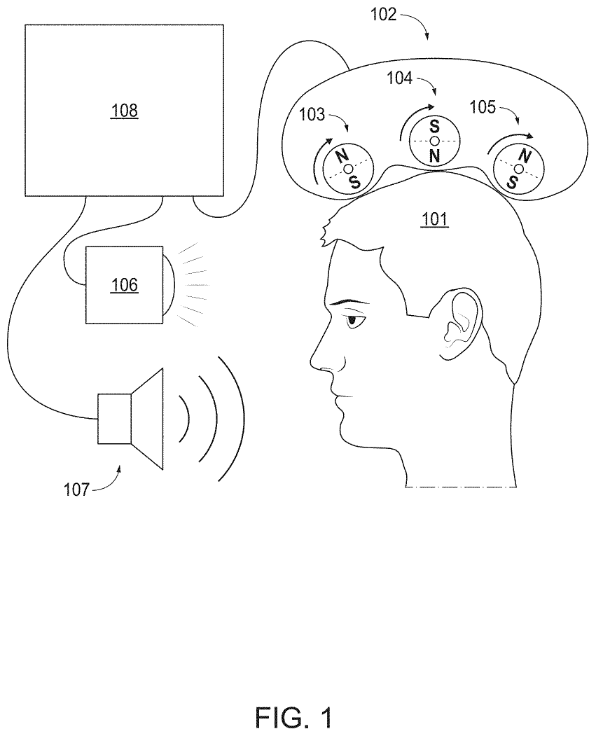

One example of a system of the present invention is shown in FIG. 1. In this example, a NEST system (102) comprising three diametrically magnetized rotating neodymium magnets (103, 104, 105) configured to be placed near a scalp of the person (101). This NEST system generates a sinusoidal magnetic field, in which the magnetic field frequency is selected to influence an intrinsic frequency of an EEG band. A light (106) is positioned in or near the visual field of the person. The light may flash on and off, or it may alternately change intensity, or it may alternately change color, or it may be a video monitor with a video image of an object moving alternately back and forth across the screen, such that the change or movement frequency is equal to, or a harmonic or subharmonic of, the frequency of the external neuromodulation. In addition, a speaker (107) is placed so that the person can hear the sound emanating from the speaker. This sound may be a tone, clicks, beeps, music, verbal, or some other means by which the sound may be made, where the sound has a modulation frequency equal to, or a harmonic or subharmonic of, the frequency of the external neuromodulation. The activation of the light, sound, and magnetic field is controlled by a Controller Unit (108), which ensures that all modalities are operating synchronously. It may also adjust the phase of each modality so that the stimulation is being given at the optimal time point. Each of the frequency of the neuromodulation, the light, and/or the speaker may operating at the same or different frequencies. For example, they may all be at the intrinsic frequency of the patient. Alternatively, one or more may be at an intrinsic frequency of the patient, while one or more others may be at a harmonic and/or subharmonic of the intrinsic frequency of the patient.

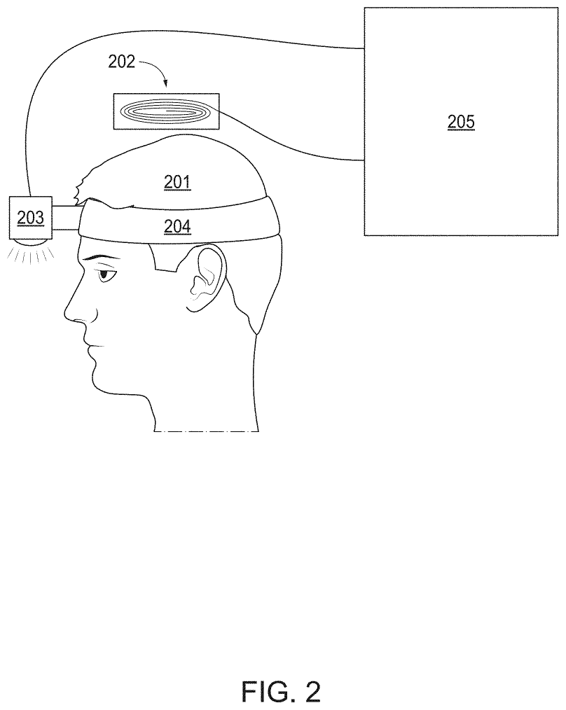

Another aspect of the present invention is shown in FIG. 2. In this example, an rTMS system using a coil (202) is positioned over the scalp of a person (201) and generates magnetic pulses at a frequency intended to influence an intrinsic frequency of an EEG band. In addition, a flashing light (203) is mounted to a headband (204), and positioned so that the flashing light is near the field of view of the person. The light flashes at a frequency approximately equal to, or a harmonic or subharmonic of, the magnetic pulse frequency. The coil and the light are controlled by a Controller Unit (205), which may control the different modalities to operate in a desired pattern or relation, such as synchronously, or sequentially.

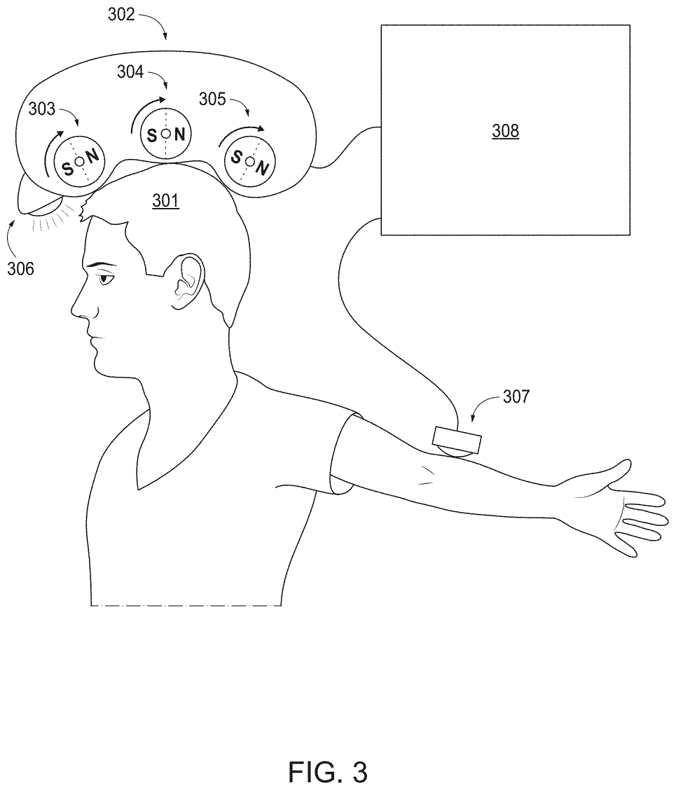

Another aspect of the present invention is shown in FIG. 3. In this, a NEST system (302) rotates three magnets (303, 304, 305) near the scalp of a person (301). A flashing light is attached to the NEST, and is electrically connected to a Controller Unit (308). A diaphragm module (307) is also connected to the Controller Unit. The diaphragm module is designed to impart a physical tapping sensation to a location on a skin of the person. The Controller Module ensures that the tapping sensation, the flashing light, and the alternating magnetic field from the NEST magnets all operate synchronously, with the light frequency and the tapping frequency equal to, or a harmonic of subharmonic of, the magnetic field frequency. To provide the tapping sensation a diaphragm may be used that is configured to move. The diaphragm may be contained in a wristband or the sleeve of a shirt or jacket. The diaphragm may be moved through mechanical device, by inflation, by pressure, etc. The light may be directed towards the eyes of the person, or the light may just be in the visual field of the person, or the light may reflect off another surface. It is often beneficial for the person to keep their eyes closed during stimulation. However, the flashing light may be positioned and be of intensity such that the light may be sensed, either consciously or subconsciously, through the eyelids. Although a diaphragm is described in this and other embodiments in this disclosure, it is contemplated that other means of imparting pressure to the skin of the subject may be used. For example, a piston, mechanical actuator, or other such device may be used in place of a diaphragm.

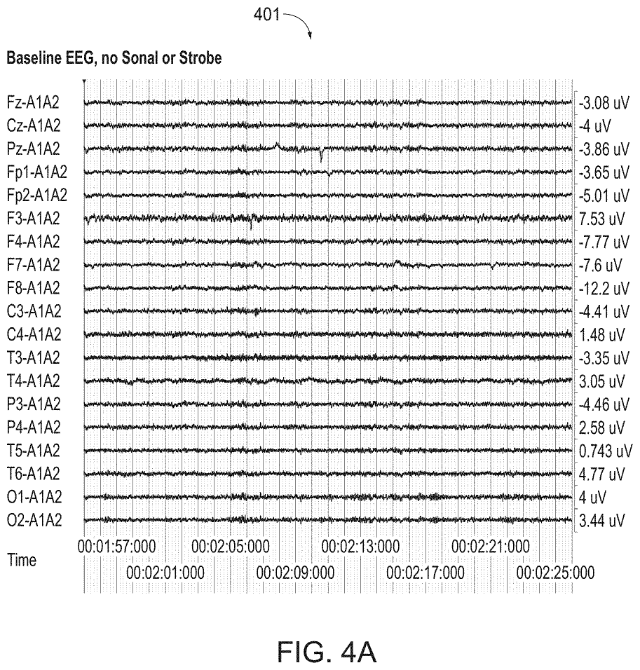

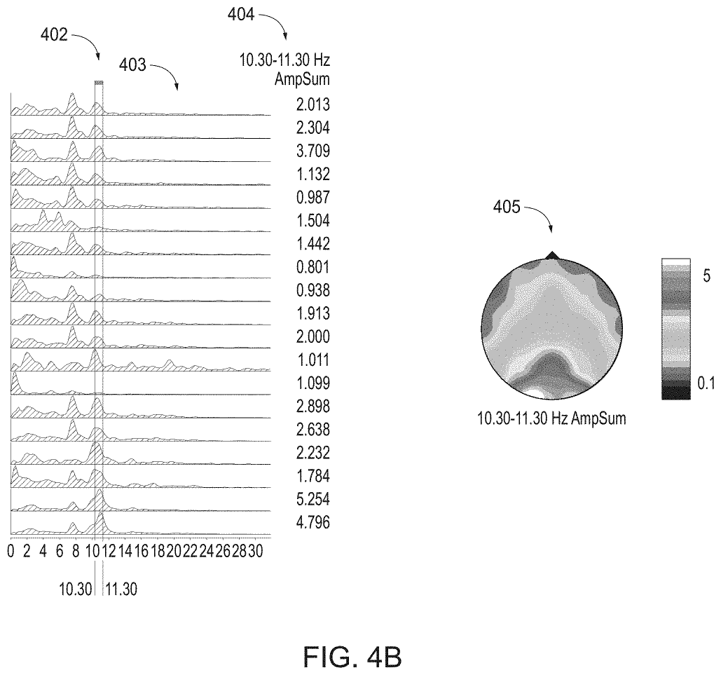

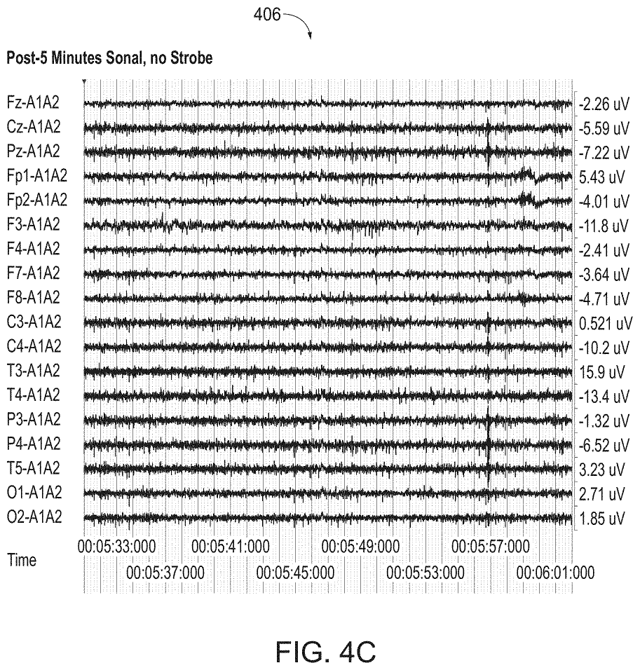

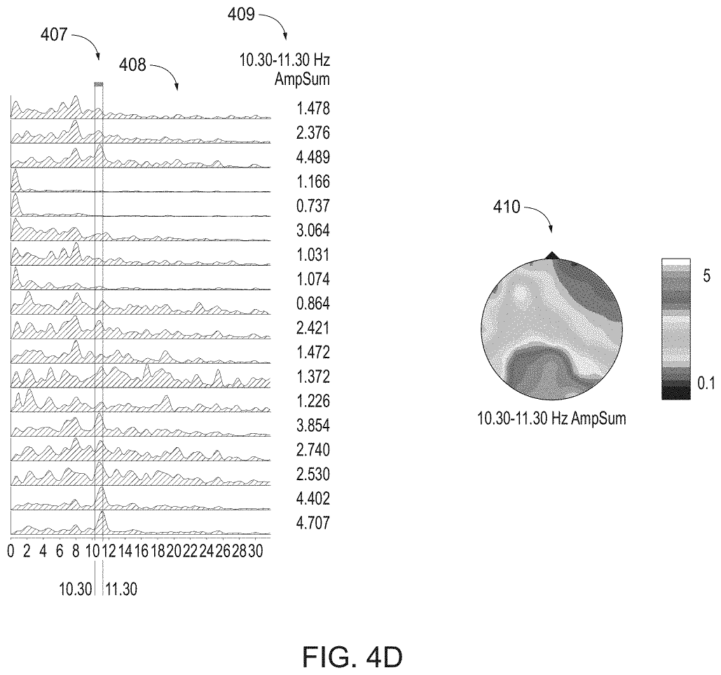

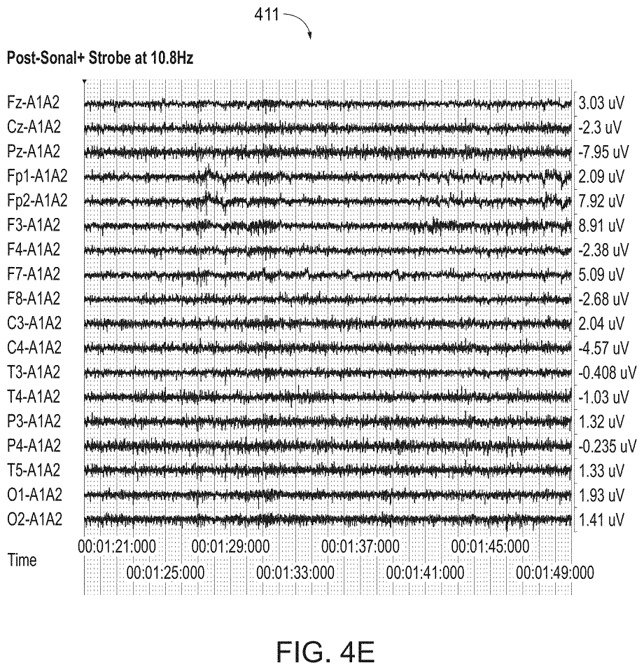

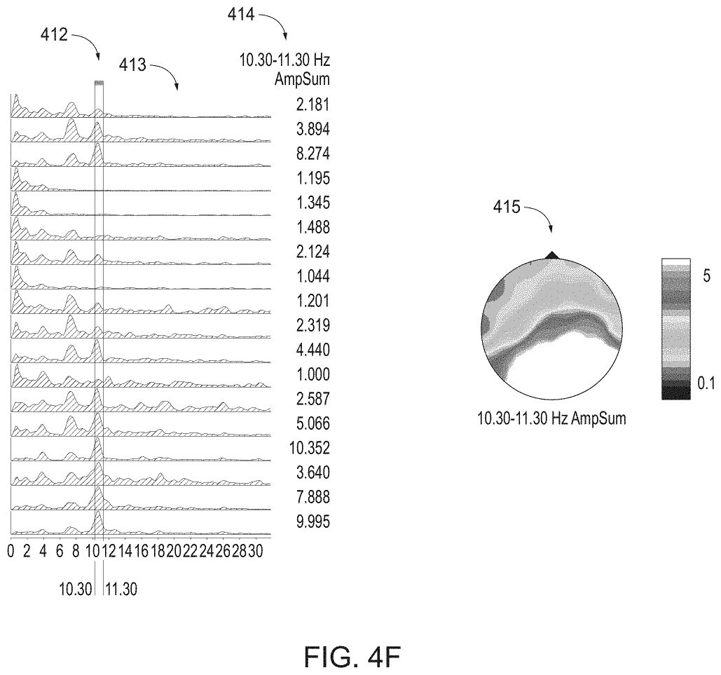

FIGS. 4A-4F show an example FFT of a User receiving NEST stimulation along with light stimulation. In FIG. 4A, the EEG (401) is shown with no NEST or light stimulation, with the frequency spectrum shown (403) in FIG. 4B. In this, the alpha frequency is shown using the cursor (402) at 10.8 Hz. A second peak at approximately 7.5 Hz can also be seen, which is outside the alpha band. The energy at the alpha frequency (404) ranges between 0.99 and 5.25, with most of the energy in the far back of the brain, as shown (405).

FIG. 4C shows the EEG (406) after the NEST has generated a magnetic field at the person's IAF for 5 minutes, but no light stimulation is provided. The EEG activity has shifted somewhat, with the activity at 7.5 Hz diminished relative to the activity at the alpha frequency (407), as shown in FIG. 4D. The energy at the alpha frequency (409) ranges between 0.74 and 4.7, and the activity has moved further forward on the head (410).

FIG. 4E shows the EEG (411) after the NEST has generated a magnetic field and a light has strobed near the person's closed lids for 5 minutes, with the magnetic field frequency and the light flashing frequency set to the person's alpha frequency (412), as shown in the frequency plot (413) in FIG. 4F. As can be seen, the energy at the alpha frequency is much more significant, with the energy at each electrode (414) ranging between 1.00 and 10.35. The alpha activity has also shifted significantly forward, as shown in the head plot (415). From these plots, it is clear that combining magnetic field stimulation with light stimulation at the person's alpha frequency results in the greatest change from baseline, where no stimulation was provided.

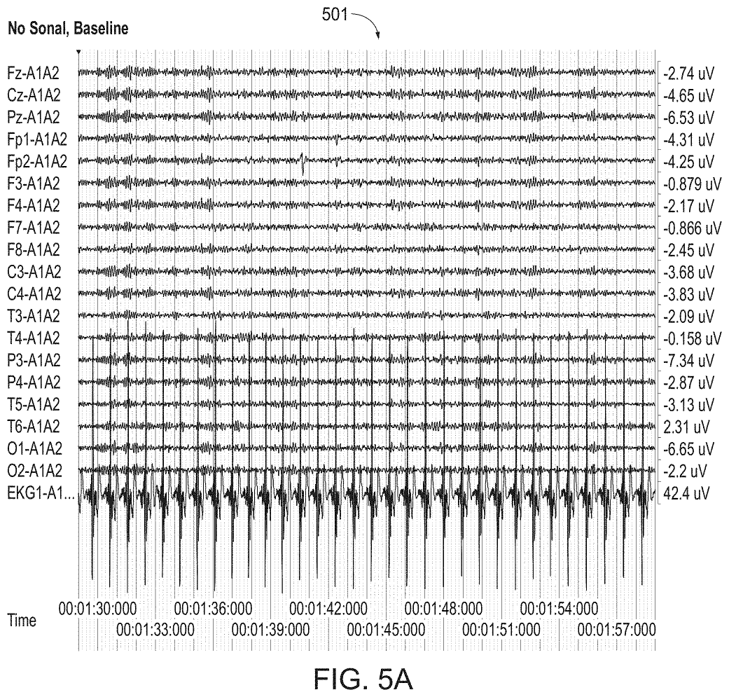

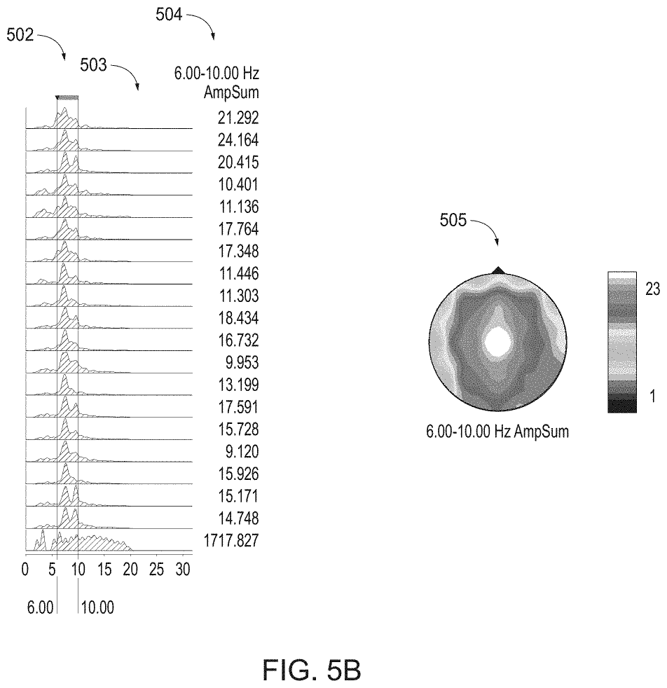

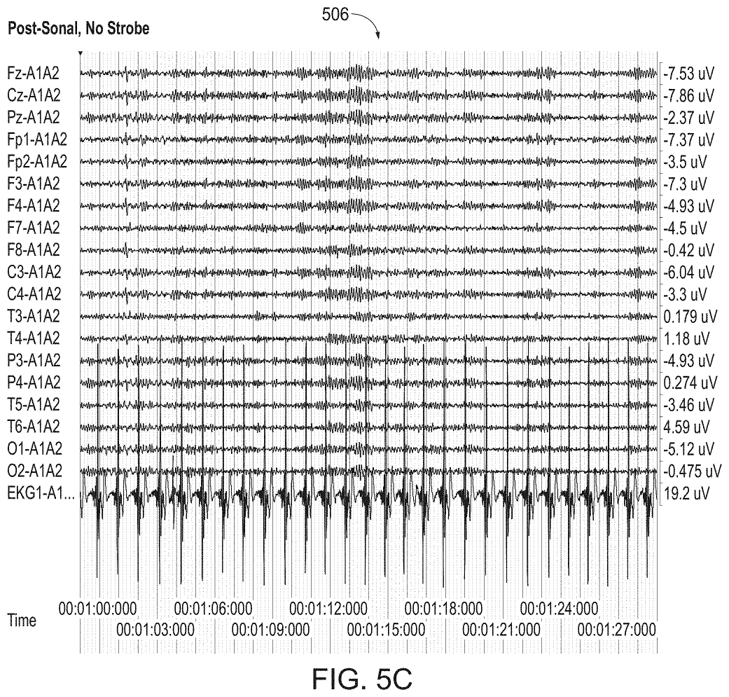

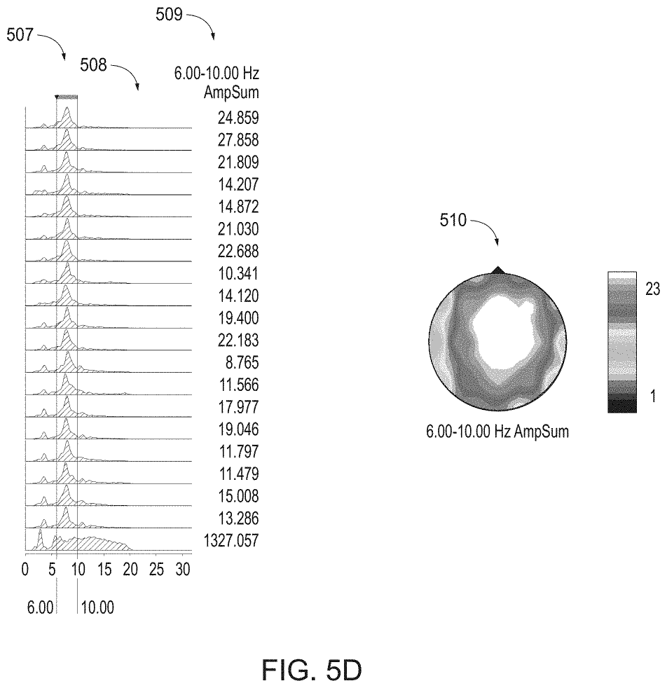

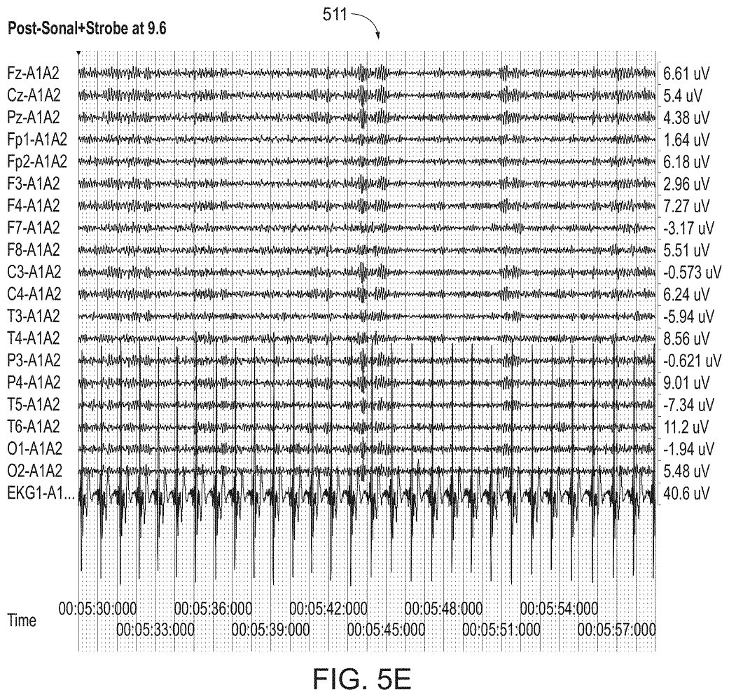

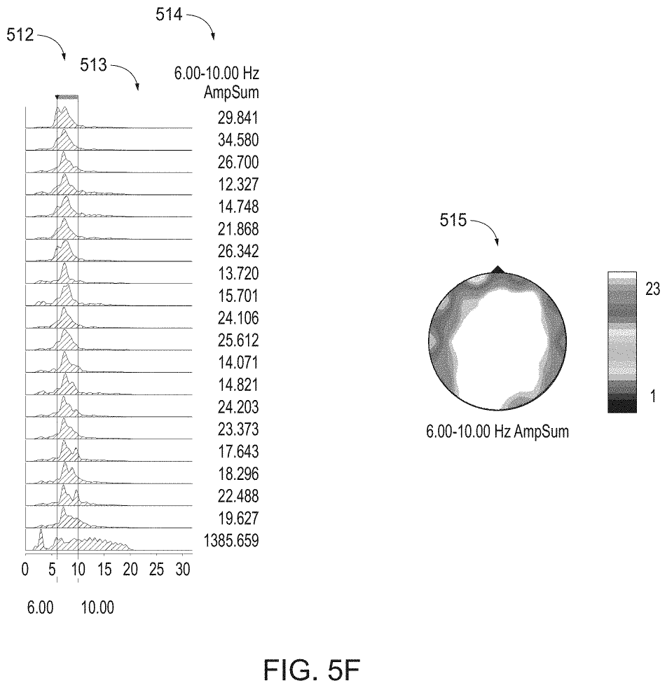

FIGS. 5A-5F show an example FFT of a different User receiving NEST stimulation along with light stimulation. In FIG. 5A, the EEG (501) is shown with no NEST or light stimulation (Baseline), with the frequency spectrum (503) shown in FIG. 5B. In this, the approximate alpha frequency is shown using the cursor (502). The selected range covers two potential peaks, one at 8.0 Hz and one at 9.6 Hz. The energy (504) in this range for different electrodes ranges between 9.12 and 24.16, with most of the energy in the center of the brain, as shown (505).

FIG. 5C shows the EEG (506) after the NEST has generated a magnetic field at 9.6 Hz for 5 minutes, but no light stimulation was provided. The frequency plot (508) of the EEG shows that the activity has become more rhythmic as seen in FIG. 5D. The NEST energy (509) between 6.0 Hz and 10.0 Hz varies between 8.75 and 27.86, and the head plot (510) shows that the energy in that range has shifted further forward.

FIG. 5E shows the EEG (511) of 5 minutes of stimulation after the NEST and the strobe light were both active at 9.6 Hz for 5 minutes. As seen in FIG. 5F, the frequency plot (513) shows the energy activity in the region of the cursor (512) generally higher, with the energy (514) varying between 13.7 and 34.58, saturating the upper limit of the measured energy range of 23. The activity in that range has shifted even farther forward, occupying nearly all of the cortex.

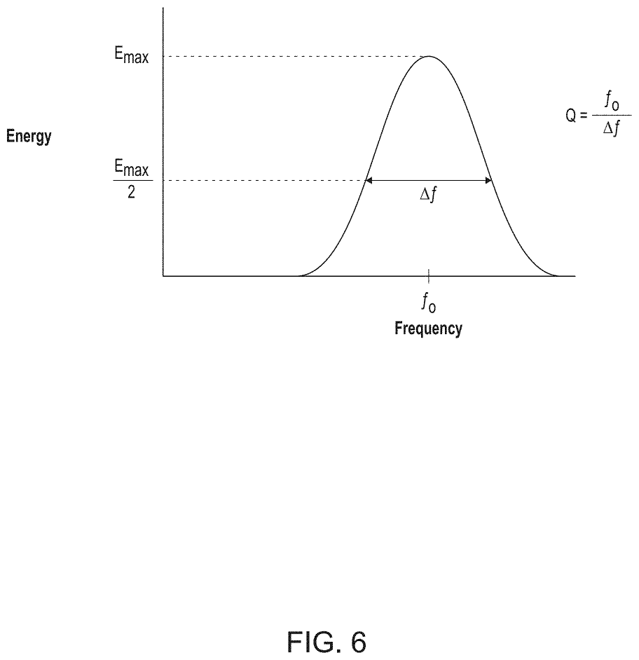

FIG. 6 shows an example of the Q-factor as used according to exemplary embodiments described herein. The figure shows a sample graph of the frequency distribution of the energy of an EEG signal. It can be seen that a frequency range, Δf can be defined as the frequency bandwidth for which the energy is above one-half the peak energy. The frequency f₀ is defined as the intrinsic frequency in the specified band. The Q-factor is defined as the ratio of f₀/Δf. As can be seen, when ΔF decreases for a given f₀, the Q-factor will increase. This can occur when the peak energy E. of the signal increases or when the bandwidth of the EEG signal decreases.

Optionally, during administration of methods described herein, the User could lie on a flat surface, such as a couch or a bed. The person preferably would be relaxed, because a relaxed state is more likely to result in the brain generating neuronal activity in the alpha EEG band. Also, relaxing keeps the muscles from contracting, which may cause artifacts to appear on the EEG that are independent of brain activity. In an example, The User could sit in a chair, or recline.

A light may be flashed with a flicker rate that matches the frequency, or a harmonic or a subharmonic of the frequency, of the neuromodulation device. The light may be flashed close to the User's eyes, or may be above the User's head, or the flashing light may be the only light in an enclosed room. In any case, the system may be configured so that the eyes of the User perceive the flashing light. In general, a User's eyes may still perceive a flashing light even if the eyes are closed, because the light may penetrate the lids.

In this example, a light is flashed. However, the most important factor in affecting neuronal activity is that an external stimulus is provided at the frequency, or a harmonic or a subharmonic of the frequency of the neuromodulation device. For example, a sound could be provided which warbles or beeps at a frequency matching the frequency, or a harmonic or a subharmonic of the frequency of the neuromodulation device. Alternately, a tapping sensation, such as by a diaphragm, would be provided on the User's skin, where the tapping frequency matches the frequency, or a harmonic or a subharmonic of the frequency of the neuromodulation device. Other or additional alternatives may include a mild electric pulse that may be given at the frequency, or a harmonic or a subharmonic of the frequency of the neuromodulation device, which is sensed by the brain.

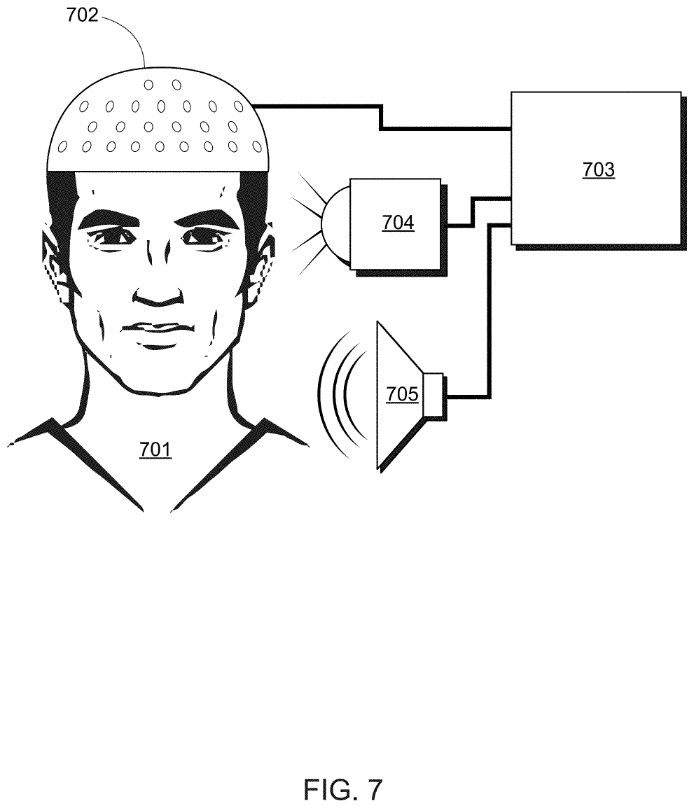

FIG. 7 shows an example system of the present invention in which a person (701) wears an EEG cap (702) comprising multiple electrodes, which is electrically connected with a processing unit (703). The processing unit comprises an EEG amplifier, a processor, and memory in order to cycle the stimulus through rhythmic stimulation frequencies (RSFs) in or around the desired EEG band, and to determine the RSF which optimizes a function (and/or metric) of the EEG recorded while the stimuli is being delivered, and uses that to estimate the intrinsic frequency of the EEG band. The rhythmic stimulus in this example is performed using a combination of a flashing light (704) and/or a speaker (705) which plays beeps, ticks, or warbling music at the RSF. The light and speaker may be positioned so that the stimulus can be sensed by the person.

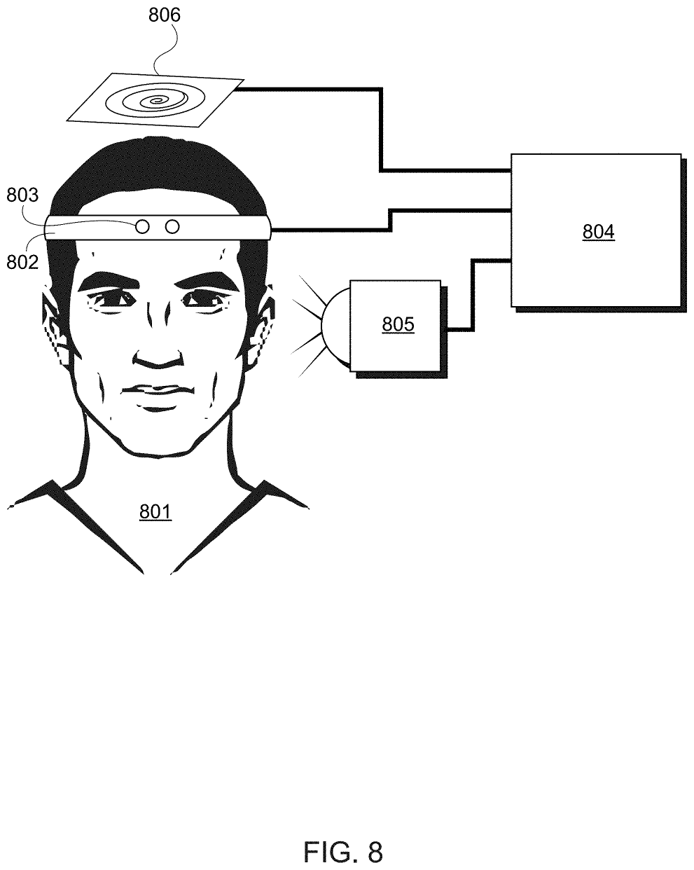

FIG. 8 shows an example system of the present invention in which a person (801) wears a headband (802) that comprises two or more EEG electrodes (803). The EEG electrodes are electrically connected to the processing unit (804), which comprises an EEG amplifier, processor, and memory to cycle through stimulus applied at rhythmic stimulation frequencies in and around the desired EEG band, and optimizes a function and/or metric of the EEG while the stimulus is being administered at the RSF across and across a set of RSF values. The estimate of the intrinsic frequency of the EEG band is based upon that optimized metric compared across the stimulus administered at a set of RSF values. The rhythmic stimulus in this example is performed using a flashing light (805). The system in this example is combined with a repetitive Transcranial Magnetic Stimulation (rTMS) coil (806), which can stimulate the brain at a pulse frequency based upon the calculated intrinsic frequency of the EEG band. Since the rTMS system also generates a physical sensation as well as brain stimulation, the rTMS pulses themselves may be used as stimuli to affect the EEG of the person, where the rTMS pulses are administered at the RSF.

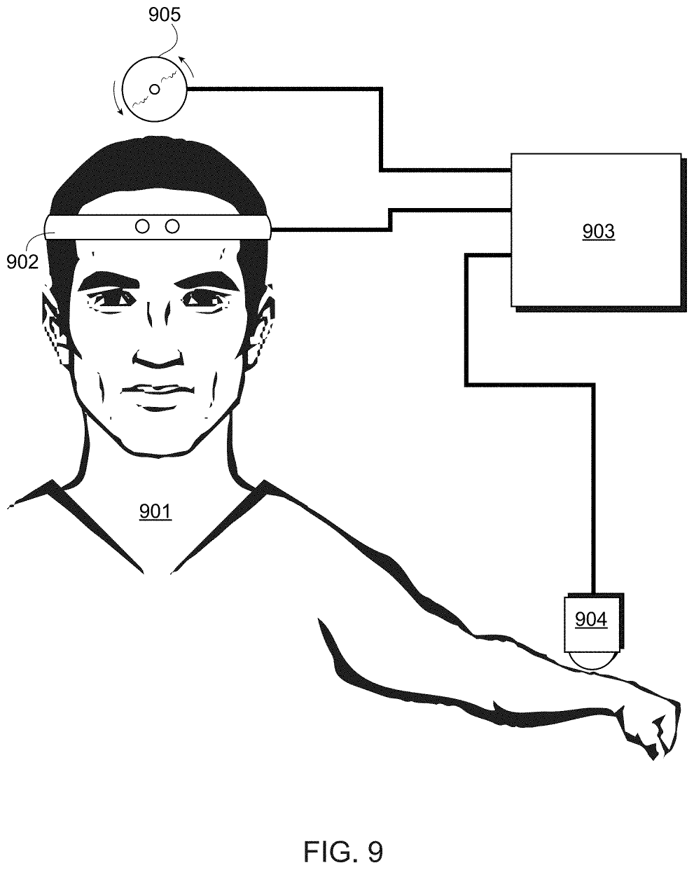

FIG. 9 shows an example system of the present invention in which a person (901) wears an EEG headband (902), which is connected electrically to a processing unit (903), which comprises an EEG amplifier, a processor, and memory in order to cycle the stimulus through RSFs in or around the desired EEG band, and to find the RSF which optimizes a function of the EEG recorded while the stimuli is being delivered, and it uses this optimized RSF to estimate the intrinsic frequency of the EEG band. The stimulus in this exampled is generated using a diaphragm unit (904), which is electrically connected to the processing unit, and where the diaphragm makes physical contact with the skin of the person. In this example, the system is combined with a magnetic field generator (905), which rotates a diametrically magnetized cylindrical magnet near the scalp of the person, in order to provide a low-level alternating magnetic field stimulation of the brain, where the cylindrical magnet is rotated at a frequency based upon the estimated intrinsic frequency of the EEG band.

Although exemplary embodiments described herein include an EEG system having two or more electrodes for sensing electrical activity of a user, the system is not so limited. Alternatively or in addition thereto, the system may include an input for receiving the intrinsic frequency of the user, and/or a target frequency in which to administer one or more modalities of stimulus according to embodiments described herein. Therefore, the EEG system may be separate from embodiments described herein. Exemplary embodiments described herein may not need an EEG analysis, but may administer the stimulus in combination at a static or given frequency, such as 10 Hz. Exemplary embodiments may also or alternatively include an interface for receiving the raw information of one or more electrodes of an EEG and may include the memory and processing to determine a frequency to administer one or more of the stimulus, such as by estimating or calculating an intrinsic frequency, and/or harmonic and/or subharmonic of the intrinsic frequency of the patient.

Exemplary embodiments described herein include a system and method for determining an intrinsic frequency of an EEG band, which comprises recording an EEG of a person. In an exemplary embodiment, the EEG band may be chosen as one which comprises an intrinsic frequency. For example, the Alpha EEG band may be the EEG band comprising the intrinsic alpha frequency (IAF). Other EEG bands are also possible in the invention. For example, the EEG band may be the Alpha, Beta, Theta, Delta, Gamma, Mu, or other EEG band. If the EEG band is the alpha band, then preferably the person will keep their eyes closed because the intrinsic alpha frequency (IAF) may be most evident on an EEG when the eyes are closed.

The User's EEG is recorded for a period of time. For example, the time may be 10.0 seconds. The time could be shorter. For example, 5.0 seconds. The time could be longer. For example, 30.0 seconds. The period of time should be sufficient so that the EEG recording time so that enough EEG can be recorded to calculate an intrinsic frequency associated with the EEG segment. If the User has a very clean artifact free EEG, then the EEG recording may be short. However, if the User has an EEG which has significant muscle artifact or is generally low energy or noisy, then a longer recording may be required. Once the EEG is recorded, a Fast Fourier Transform (FFT) of the EEG waveform may be calculated. At this point, the algorithm may select an estimate of the IAF.

Exemplary embodiments described herein may administer the one or more stimuli at the IAF of the patient.

Exemplary embodiments described herein include providing a rhythmic stimulus at a specified frequency to the person. The stimulus may be light (flashing light, video, changing colors or intensity, etc.), sound, touch, air pressure, vibration, electric current (transcutaneous, induced electromagnetic, etc.), or any other stimulus which may be sensed, either consciously or unconsciously, by the person. The stimulus may also be a combination of stimuli. For example, combining light and sound.

Exemplary embodiments of a sensed stimulus may include, for example, a stimulus that is consciously perceived by a patient, through one or more of the patient's senses. Exemplary sensed stimulus may be through tactile sense administered through contact or touch, may be through visual sense such as administered through light or other visual, may be through audio sense administered through sound.

Exemplary embodiments of a stimulus that is not sensed may include, for example, a stimulus that has a stimulus effect on the patient that is unknown to the patient or not received through one of the conventional five senses. A non-sensed stimulus may be a magnetic field, electric field, current, etc.

The patient should preferably be calm and relaxed when the rhythmic stimulus is generated. If the stimulus is a flashing light, it should preferably be placed where the person can sense when the light flashes. For example, a strobe light may be placed within the person's field of vision. If the patient's eyes are closed, although not required, then a flashing light should be strong enough so that it can be sensed by the eyes through closed eyelids. If the stimulus is sound, the person could wear headphones and listen for an audible beep, warble, or other rhythmically varying audible signal. If the stimulus is touch, then the person could wear a band that comprises a diaphragm, which presses on the person's skin in a rhythmic manner.

The User may be aware of the stimulus, or the user may be unaware of the stimulus. For example, if the stimulus level is very low, it may influence the patient but still be unnoticed by the person. Alternately, the person could be distracted by some means so they are not aware of the stimulus. For example, the User could listen to music. If audio stimulus is administered at a specific frequency and incorporated into the music, it may not be noticed by the User.

Exemplary systems described herein may be combined with a repetitive Transcranial Magnetic Stimulation (rTMS) coil, which can stimulate the brain at a pulse frequency based upon the calculated intrinsic frequency of the EEG band.

Exemplary systems described herein may be combined with a magnetic field generator, which rotates a diametrically magnetized cylindrical magnet near the scalp of the person, in order to provide a low-level alternating magnetic field stimulation of the brain, where the cylindrical magnet is rotated at a frequency based upon the estimated intrinsic frequency of the EEG band.

While the present disclosure has been described with reference to exemplary embodiments, it is to be understood that the invention is not limited to the disclosed exemplary embodiments. The scope of the following claims is to be accorded the broadest interpretation to encompass all modifications, equivalent structures, and functions.

Exemplary embodiments of the system described herein can be based in software and/or hardware. While some specific embodiments of the invention have been shown the invention is not to be limited to these embodiments. For example, most functions performed by electronic hardware components may be duplicated by software emulation. Thus, a software program written to accomplish those same functions may emulate the functionality of the hardware components in input-output circuitry. The invention is to be understood as not limited by the specific embodiments described herein, but only by scope of the appended claims.

As used herein, the terms “about,” “substantially,” or “approximately” for any numerical values, ranges, shapes, distances, relative relationships, etc. indicate a suitable dimensional tolerance that allows the part or collection of components to function for its intended purpose as described herein. Numerical ranges may also be provided herein. Unless otherwise indicated, each range is intended to include the endpoints, and any quantity within the provided range. Therefore, a range of 2-4, includes 2, 3, 4, and any subdivision between 2 and 4, such as 2.1, 2.01, and 2.001. The range also encompasses any combination of ranges, such that 2-4 includes 2-3 and 3-4.

Although embodiments of this invention have been fully described with reference to the accompanying drawings, it is to be noted that various changes and modifications will become apparent to those skilled in the art. Such changes and modifications are to be understood as being included within the scope of embodiments of this invention as defined by the appended claims. Specifically, exemplary components are described herein. Any combination of these components may be used in any combination. For example, any component, feature, step or part may be integrated, separated, sub-divided, removed, duplicated, added, or used in any combination and remain within the scope of the present disclosure. Embodiments are exemplary only, and provide an illustrative combination of features, but are not limited thereto. For example, any combination of systems and features for applying a stimulus, monitoring the EEG when a stimulus is being applied, and/or the methods and systems for estimating an intrinsic alpha frequency may be used with any system for administering a magnetic field to the patient at the estimated intrinsic alpha frequency. In addition, and combination of stimuli may be used in combination and/or any combination of algorithms to determine one or more metrics and/or optimize the one or more metrics may be used and are within the scope of the instant disclosure.

When used in this specification and claims, the terms “comprises” and “comprising” and variations thereof mean that the specified features, steps, or integers are included. The terms are not to be interpreted to exclude the presence of other features, steps, or components.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be utilized for realizing the invention in diverse forms thereof.

What is claimed is:

1. A device comprising:
at least one permanent magnet having a rhythmic movement with a rhythmic movement frequency;
at least one light having a rhythmically changing intensity with an intensity frequency;
a controller coupled to the magnet and to the light;
wherein the controller comprises a processor that controls the rhythmic movement frequency of said at least one magnet between about 0.5 Hz and 100 Hz based on an intrinsic frequency of a brain of a subject within a specified EEG band; and controls the intensity frequency of at least one light set about equal to the rhythmic movement frequency, or a harmonic or subharmonic frequency thereof.

2. The device of claim 1, wherein the at least one permanent magnet is a rotating permanent magnet.

3. The device of claim 1, wherein the at least one permanent magnet comprises three diametrically magnetized permanent magnets.

4. The device of claim 1, wherein the at least one light produces a visible signal in the form of at least one of a flashing light, alternating light intensity, alternating light color, and rhythmic video images.

5. The device of claim 1, further comprising at least one speaker coupled to the controller, wherein the speaker is capable of creating sounds with a modulation frequency equal to the rhythmic movement frequency, or a harmonic or subharmonic frequency thereof.

6. The device of claim 1, further comprising at least one component coupled to the controller to apply pressure to the subject at the rhythmic movement frequency, or a harmonic or subharmonic frequency thereof

7. A device comprising:
at least one magnetic field generator having a programmable pulse frequency;
at least one light having a rhythmically changing intensity with an intensity frequency; and
a controller coupled to the pulse generator and to the light;
wherein the controller comprises a processor that controls the pulse frequency between about 0.5 Hz and 100 Hz based on an intrinsic frequency of a brain of a subject within a specified EEG band; and controls the intensity frequency of at least one light set about equal to the pulse frequency, or a harmonic or subharmonic thereof.

8. The device of claim 7 wherein the magnetic field generator is a Transcranial Magnetic Stimulation (TMS) coil.

9. The device of claim 7, wherein the magnetic field generator is a rotating permanent magnet.

10. The device of claim 9 wherein the magnetic field generator comprises one or more diametrically magnetized cylindrical magnet configured to rotate near the scalp of the subject.

11. The device of claim 7, wherein the at least one light produces a visible signal in the form of at least one of a flashing light, alternating light intensity, alternating light color, and rhythmic video images.

12. The device of claim 7, further comprising at least one speaker coupled to the controller, wherein the speaker is capable of creating sounds with a modulation frequency equal to the pulse frequency, or a harmonic or subharmonic thereof.

13. The device of claim 7, further comprising at least one component coupled to the controller to apply pressure to the subject at the pulse frequency, or a harmonic or subharmonic frequency thereof.

14. A device comprising:
a controller comprising a processor, an EEG amplifier, and memory;
a first electrode configured to detect electrical brain activity and coupled to the controller;
a second electrode configured to detect a reference signal and coupled to the controller;
wherein the controller is configured to generate and provide a stimulus to subject through rhythmic stimulation frequencies in or around a desired EEG band; determine an optimal rhythmic stimulation frequency which optimizes an aspect of an EEG spectrum recorded while the stimulus is being delivered; estimate the intrinsic frequency of the EEG band based on EEG data received from the subject.

15. The device of claim 14, wherein the stimulus is in the form of visible signals generated by one or more lights coupled to the controller.

16. The device of claim 14, wherein the stimulus is in the form of a sound generated by one or more speakers coupled to the controller.

17. The device of claim 14, wherein the stimulus is in the form of pressure applied to the skin of the subject.

18. The device of claim 14, wherein the first and second electrodes are part of an EEG cap.

19. The device of claim 14, wherein the first and second electrodes are part of a headband.
